(19) United States
(12) Patent Application Publication
(10) Pub. No.: US 2025/0128051 A1
(43) Pub. Date: Apr. 24, 2025

(54) MECHANICAL CIRCULATORY SUPPORT SYSTEM WITH REPOSITIONING SHEATH

(71) Applicants: Boston Scientific Scimed, Inc., Maple Grove, MN (US); Boston Scientific Medical Device Limited, Galway (IE)

(72) Inventors: Qian Liu, Plymouth, MN (US); Neeraj Kumar Sharma, Delhi (IN)

(73) Assignees: Boston Scientific Scimed, Inc., Maple Grove, MN (US); Boston Scientific Medical Device Limited, Galway (IE)

(21) Appl. No.: 18/917,549

(22) Filed: Oct. 16, 2024

Related U.S. Application Data
(60) Provisional application No. 63/591,814, filed on Oct. 20, 2023.

(51) Int. Cl. A61M 60/865; A61M 25/00; A61M 25/01; A61M 25/06; A61M 39/06; A61M 60/13; A61M 60/17

(52) U.S. Cl. CPC A61M 60/865; A61M 25/0097; A61M 25/0102; A61M 39/06; A61M 60/13; A61M 60/17; A61M 2025/0681; A61M 2039/062

(57) ABSTRACT

A mechanical circulatory support system may include a blood pump, a housing, an elongate shaft, a first sheath, and a second sheath. The blood pump may be configured to pump blood from a ventricle of a heart of a patient to vasculature of the patient. The elongate shaft may be coupled with the blood pump and extend proximally from the blood pump to the housing. The first sheath may extend over the elongate shaft distal of the housing and include a lumen with an inner diameter. The second sheath may extend over the elongate shaft distal of the housing and include an outer diameter that is less than the inner diameter of the lumen of the first sheath. The first sheath and the second sheath are configured to longitudinally adjust over the elongate shaft and an entirety of the second sheath is configured to adjust through the first sheath.

### CROSS-REFERENCE TO RELATED APPLICATIONS

The present application claims the benefit of U.S. Provisional Patent Application Ser. No. 63/591,814, filed Oct. 20, 2023, the disclosure of which is incorporated herein by reference.

### TECHNICAL FIELD

The present disclosure pertains to a sheath configuration for a mechanical circulatory support system. More particularly, the present disclosure pertains to a repositioning sheath of a mechanical circulatory support system for use at a vascular access site.

### BACKGROUND

In various procedures for delivering intravascular medical devices, an introducer sheath is inserted into a blood vessel of a patient, for example a femoral artery, and one or more medical devices may be advanced through the sheath and into the patient's vasculature. In various instances, the medical devices include catheters or other devices, such as a blood pump. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### BRIEF SUMMARY

This disclosure provides design, material, manufacturing method, and use alternatives for medical devices, including percutaneous blood pumps and associated devices.

A first example may include a mechanical circulatory support system comprising a blood pump configured to pump blood from a ventricle of a heart of a patient to vasculature of the patient, a housing, an elongate shaft coupled with the blood pump and extending proximally from the blood pump to the housing, a first sheath configured to extend over the elongate shaft distal of the housing, the first sheath has a lumen with an inner diameter, and a second sheath configured to extend over the elongate shaft distal of the housing, the second sheath has an outer diameter that is less than the inner diameter of the lumen, wherein the first sheath and the second sheath may be configured to longitudinally adjust over the elongate shaft and an entirety of the second sheath may be configured to adjust through the first sheath.

Alternatively or additionally to any of the examples above, the first sheath may be an introducer sheath configured to allow the blood pump to pass through the lumen.

Alternatively or additionally to any of the examples above, the second sheath may be a dual lumen sheath.

Alternatively or additionally to any of the examples above, the dual lumen sheath may comprise a first tube defining a first lumen, a second tube extending along the first tube and defining a second lumen, and a third tube extending over the first tube and the second tube.

Alternatively or additionally to any of the examples above, the system may further include a wire, wherein the dual lumen sheath may define a first lumen configured to receive the elongate shaft and a second lumen configured to receive the wire.

Alternatively or additionally to any of the examples above, the second sheath may include an internal hemostasis valve.

Alternatively or additionally to any of the examples above, the system may further include a hub assembly configured to couple with a proximal end of the second sheath.

Alternatively or additionally to any of the examples above, the system may further include a wire with a connector at a proximal end of the wire, the connector is configured to couple with the hub assembly.

Alternatively or additionally to any of the examples above, the hub assembly may be configured to releasably engage the elongate shaft to fix the second sheath with respect to the elongate shaft.

Alternatively or additionally to any of the examples above, the system may further include a sleeve extending over the elongate shaft distal of the housing, the sleeve has a distal end configured to couple with the hub assembly.

In a further example, a method of adjusting a tube assembly for a mechanical circulatory support system may comprise advancing a blood pump and an elongate shaft extending proximally from the blood pump through a first sheath, advancing the first sheath along the elongate shaft in a proximal direction, advancing an entirety of a second sheath through the first sheath, and coupling a hub assembly with a proximal end of the second sheath.

Alternatively or additionally to any of the examples above, the method may further comprise coupling the hub assembly with the elongate shaft and advancing the hub assembly coupled with the elongate shaft toward the proximal end of the second sheath to couple the hub assembly with the proximal end of the second sheath.

Alternatively or additionally to any of the examples above, the second sheath may have a first lumen configured to receive the elongate shaft and a second lumen and the method may further comprise inserting a wire through the second lumen.

Alternatively or additionally to any of the examples above, the method may further comprise coupling a connector at a proximal end of the wire to the hub assembly.

Alternatively or additionally to any of the examples above, the method may further comprise coupling the hub assembly with the elongate shaft, releasing the hub assembly from the elongate shaft, adjusting the elongate shaft relative to the hub assembly and the second sheath, and re-coupling the hub assembly with the elongate shaft.

Alternatively or additionally to any of the examples above, the method may further comprise passing a distal end of a sleeve through the first sheath and coupling the distal end of the sleeve with the hub assembly.

Alternatively or additionally to any of the examples above, the method may further comprise adjusting the elongate shaft relative to the hub assembly and the second sheath, and wherein adjusting the elongate shaft may comprise engaging the elongate shaft through the sleeve and advancing the elongate shaft proximally or distally.

In a further example, a mechanical circulatory support system may comprise a blood pump configured to pump blood from a ventricle of a heart of a patient to vasculature of the patient, an elongate shaft coupled with the blood pump and extending proximally from the blood pump, a repositioning sheath extending over the elongate shaft, and a hub assembly configured to couple to a proximal end of the repositioning sheath, wherein the hub assembly may be configured to lock the repositioning sheath at a position relative to the elongate shaft.

Alternatively or additionally to any of the examples above, the system may further comprise a sterile sleeve extending over the elongate shaft, wherein the hub assembly may be configured to couple with a distal end of the sterile sleeve.

Alternatively or additionally to any of the examples above, the system may further comprise a stylet having a connector and a wire extending distally from the connector, and wherein the repositioning sheath may have a first lumen configured to receive the elongate shaft and a second lumen configured to receive the wire and the hub assembly may be configured to couple with the connector.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a schematic perspective view of an illustrative catheter system including a percutaneous blood pump;

FIG. 2 is a schematic partial cross-section view of a proximal end of an illustrative catheter system, in a first configuration;

FIG. 3 is a schematic partial cross-section view of the proximal end of the illustrative catheter system depicted in FIG. 1, in a second configuration;

FIG. 4 is a schematic cross-section view of an illustrative repositioning sheath;

FIG. 5 is a schematic cross-section view of an illustrative repositioning sheath with a hub and positioned on an elongate shaft;

FIGS. 6A-6H depict a schematic flow of an illustrative technique for adjusting a tube assembly for a catheter system;

FIGS. 7A-7C depict a schematic flow of an illustrative technique for adjusting a tube assembly for a catheter system;

FIGS. 8A and 8B depict a schematic flow of an illustrative technique for adjusting a tube assembly for a catheter system; and

FIGS. 9A-9G depict a schematic flow of an illustrative technique for adjusting a tube assembly for a catheter system.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used connection with other configurations whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

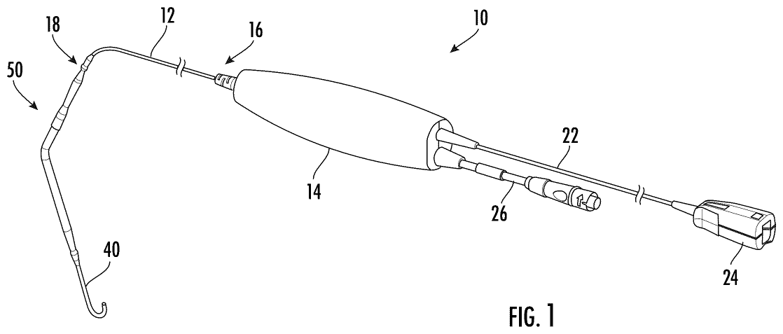

FIG. 1 illustrates a perspective view of a catheter 10 (e.g., a mechanical circulatory support system and/or other suitable catheter system) including a percutaneous blood pump 50 located at a distal end region thereof. Although not required, the blood pump 50 may be configured to pump blood from a ventricle of a heart of a patient to vasculature of the patient.

The catheter 10 may be coupled to or may include the blood pump 50, with an elongate shaft 12 (e.g., an elongate tube with one or more elongate members extending therein) of the catheter 10 coupled with and extending proximally from the percutaneous blood pump 50 and a distal tip 40 extending distally from the blood pump 50. For instance, a proximal end 16 of the elongate shaft 12 may be coupled to a housing 14 (e.g., a housing of a handle, of a control module, etc.) and a distal end 18 of the elongate shaft 12 may be coupled to the percutaneous blood pump 50. An electrical cable 22 may extend from the housing 14 to a connector 24 at a proximal end thereof. The connector 24 may be configured to be connected to a controller (not shown) for controlling the blood pump 50, such as providing electrical power and/or control signals to the blood pump 50. The catheter 10 may also include an extension 26 connectable to the controller for sending and/or receiving signals, such as from one or more sensors during operation of the blood pump 50.

When placing the blood pump 50 in a subject (e.g., a patient), the blood pump 50 may be inserted through an introducer sheath (e.g., a first sheath) inserted into a vascular access site of the subject. In some cases, the introducer sheath may be a large bore sheath configured to receive and facilitate the passage of the blood pump 50 and the elongate shaft 12 through a lumen thereof and into a blood vessel (e.g., a femoral artery, etc.) of the subject. Such an introducer sheath, however, may have an outer diameter that tends to block blood flow through the blood vessel (e.g., flow through the femoral artery to a leg of the subject).

To facilitate perfusion of blood to the leg and/or other portions of the subject, the introducer sheath may be swapped out for or replaced with a smaller outer diameter sheath, such as a repositioning sheath (e.g., a second sheath), after advancing the blood pump 50 into the vasculature of the subject. To facilitate removal of the introducer sheath from the vascular access site, the introducer sheath may be a peel-away sheath, where the introducer sheath may be configured to split along a perforated line extending along an entirety of or at least part of a length of the introducer sheath, and/or other suitable sheaths or tubes removable from the catheter 10 in one or more other suitable manners.

Peel-away introducer sheaths may be undesirable for a variety of reasons. In some examples, peel-away introducer sheaths may unintentionally or uncontrollably peel, which may lead to vessel trauma and/or other issues. Further, peel-away introducer sheaths may be formed from a polymer, which may require a thicker wall than other materials (e.g., metals) used for forming the introducer sheath to achieve a desired column strength along the introducer sheath. As such, a polymer peel-away introducer sheath is used, a larger hole in the patient may be required for receiving the introducer sheath relative to when a non-peel-away introducer sheath is utilized.

The concepts discussed herein include utilizing an introducer sheath and a repositioning sheath that are both configured to receive and adjust over (e.g., longitudinally or axially adjust over) the elongate shaft 12. In some configurations, the repositioning sheath may be configured to extend entirely through the introducer sheath (e.g., where the repositioning sheath is fully or entirely distal of a distal end of the introducer sheath) so as to insert into the access site of the subject with the introducer sheath at or withdrawn from the access site.

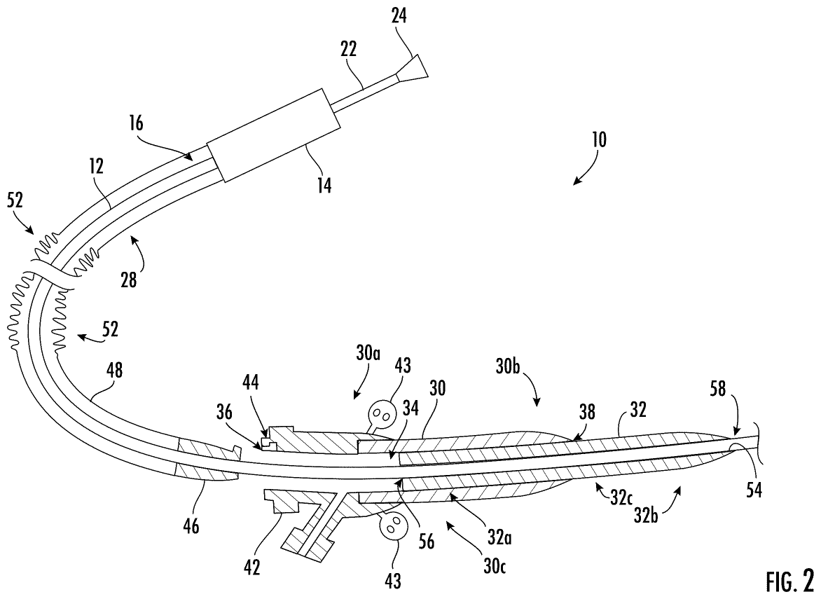

FIG. 2 depicts a schematic partial cross-section view of an illustrative configuration of a proximal end of the catheter 10. The proximal end of the catheter 10 may include the elongate shaft 12 extending distally from the housing 14 through a sterile sleeve 28, an introducer sheath 30, and a repositioning sheath 32, wherein the sterile sleeve 28, the introducer sheath 30, and the repositioning sheath 32 are depicted in cross-section.

In some cases, the introducer sheath 30 may be used for facilitating passage of various medical devices, such as the elongate shaft 12, the blood pump 50, etc., through an access site of the subject and into a blood vessel of the subject. In some examples, the introducer sheath 30 may be configured to extend into the blood vessel of the subject and allow the elongate shaft 12, the blood pump 50, and/or other suitable medical devices to pass through.

The introducer sheath 30 may include a proximal end region 30a proximate a proximal end of the introducer sheath 30 and a distal end region 30b proximate a distal end of the introducer sheath 30. A body region 30c of the introducer sheath 30 may extend between the proximal end region 30a and the distal end region 30b, where one or more of the proximal end region 30a, the distal end region 30b, and the body region 30c may define a lumen 34 of the introducer sheath 30. Further, the introducer sheath 30 may include a proximal opening 36 at or proximate the proximal end region 30a and a distal opening 38 at or proximate the distal end region 30b, with the lumen 34 extending between the proximal opening 36 and the distal opening 38.

The introducer sheath 30 may be formed from one or more suitable materials. Example suitable materials include, but are not limited to, polymeric and/or metallic materials. In some instances, the introducer sheath 30 may include one or more additional surface coatings on the interior surface defining the lumen 34 and/or the exterior surface, which may include, but are not limited to, silicone, polyethylene terephthalate (PET), and/or other suitable coating materials.

The introducer sheath 30 (e.g., at the proximal end region thereof) may include a hub 42 (e.g., a hub assembly such as hemostasis valve hub and/or other suitable hub assembly) that may provide access to the lumen 34. The hub 42 may be configured for hemostasis by, for example, preventing or mitigating blood leakage out of the introducer sheath 30 during use of the introducer sheath 30. For example, a medical device, such as the elongate shaft 12 and/or the blood pump 50, may be inserted through the hub 42 and the lumen 34 of the introducer sheath 30 and into the blood vessel of the subject, and the hub 42 may maintain hemostasis between the medical device, the introducer sheath 30, and the external surroundings.

After advancing the medical device through the introducer sheath 30 and positioning the medical device at a target site within the subject, fixation of the axial and radial position of the medical device may be desired to ensure that the medical device (and any device coupled thereto) is maintained in a proper position during use. It may also be desired for the medical personnel to reposition the medical device after insertion. As such, the hub 42 may include an adjustable tightening port 44, provided with or attachable thereto, that provides for the selectable fixation of the medical device (e.g., the elongate shaft 12) with respect to the hub 42. To affix the sheath relative to the access site of the subject, the hub 42 may include one or more suture pads 43 (e.g., two suture pads 43 extending from the hub 42, as depicted in FIG. 2) that may be sutured and/or otherwise coupled to a skin surface of the subject.

The tightening port 44 of the hub 42 may be any suitable type of tightening port. For example, the tightening port 44 may tighten around and/or release from the elongate shaft 12 in response to rotating a rotational actuator of the tightening port 44, adjusting a clip of the tightening port 44, and/or the tightening port 44 may adjust about the elongate shaft 12 in one or additional or alternative manners.

The repositioning sheath 32 may be configured to replace the introducer sheath 30 at the access site and may be configured to extend and/or advance over the elongate shaft 12 and through the introducer sheath 30 to the access site of the subject. For example, once the blood pump 50 has been positioned in the blood vessel of the subject and/or at a target site within the subject, the repositioning sheath 32 may be advanced toward the access site through the introducer sheath 30 and positioned across the access site and in the blood vessel of the subject. The repositioning sheath 32 may be inserted into the access site and the blood vessel of the subject before, during, and/or after the introducer sheath 30 has been removed from the access site.

When positioned in the blood vessel of the subject, the repositioning sheath 32 may facilitate repositioning of the blood pump 50 at or proximate the target site within the subject through adjustment of the elongate shaft 12 extending into the access site of the subject and the blood vessel via the repositioning sheath 32. The repositioning sheath 32 may facilitate increased blood perfusion through the blood vessel relative to the blood perfusion that occurs when the introducer sheath 30 is in the blood vessel due, at least in part, to the repositioning sheath 32 having a smaller outer diameter than the introducer sheath 30.

The repositioning sheath 32 may have a shaft configuration and include a proximal end region 32a proximate a proximal end of the repositioning sheath 32 and a distal end region 32b proximate a distal end region of the repositioning sheath 32. A body region 32c of the repositioning sheath 32 may extend between the proximal end region 32a and the distal end region 32b, where one or more of the proximal end region 32a, the distal end region 32b, and the body region 32c may define a lumen 54 of the repositioning sheath 32. Further, the repositioning sheath 32 may include a proximal opening 56 at or proximate the proximal end region 32a and a distal opening 58 at or proximate the distal end region 32b, with the lumen 54 extending between the proximal opening 56 and the distal opening 58.

The repositioning sheath 32 may have any suitable number of lumens. Although FIG. 2 depicts only the single lumen 54 of the repositioning sheath 32, the repositioning sheath may have a single lumen, two lumens, three lumens, and/or other suitable number of lumens as desired.

The repositioning sheath 32 may be formed in any suitable manner. In some examples, the repositioning sheath 32 may be formed with one or more extrusion processes and/or other suitable manufacturing processes. When the repositioning sheath 32 is formed with more than one lumen, the different lumens may be formed by combining multiple tubes within an outer tube, but this is not required and other configurations for forming multiple lumens are contemplated. In one example, when the repositioning sheath 32 includes two lumens, a first lumen may be formed from a first tube, a second lumen may be formed from a second tube, and a third tube may surround the first tube and the second tube to form the outer surface and/or diameter of the repositioning sheath 32. The tubes of the repositioning sheath 32, when multiple tubes are utilized, may be coupled using any suitable coupling technique including, but not limited to, adhesives, reflow techniques, and/or other suitable coupling techniques.

The repositioning sheath 32 may be formed from one or more suitable materials. Example suitable materials include, but are not limited to, polymeric and/or metallic materials. In some instances, the repositioning sheath 32 may include one or more additional surface coatings on the interior surface defining the lumen 54 and/or the exterior surface, which may include, but are not limited to, silicone, polyethylene terephthalate (PET), and/or other suitable coating materials.

The repositioning sheath 32 may have any suitable outer diameter that is smaller than the outer diameter of the introducer sheath 30 so as to better allow blood perfusion through the blood vessel of the subject in which the elongate shaft 12 is inserted and reduce a maintained diameter opening at the access site of the subject while the blood pump 50 is positioned at a target site (e.g., during a dwell time). In some examples, the outer diameter of the repositioning sheath 32 may be less than an inner diameter of the introducer sheath 30 to facilitate the repositioning sheath 32 advancing (e.g., in a proximal and/or distal axial direction) through the introducer sheath 30, but this is not required and the introducer sheath 30 may be configured to stretch or flex radially outward to allow the repositioning sheath 32 to pass through the introducer sheath 30.

The repositioning sheath 32 may be configured to be positioned entirely distal of the introducer sheath 30 (e.g., such that a proximal end of the repositioning sheath 32 is distal of a distal end of the introducer sheath 30). In such instances, prior to positioning a proximal end of the repositioning sheath 32 distal of the distal end of the introducer sheath, the repositioning sheath 32 may be free from having a hub as an entirety of the repositioning sheath 32 may advance distally through the introducer sheath 30. However, the proximal end of the repositioning sheath 32 may be configured to couple with a hub, as discussed below or otherwise.

The sterile sleeve 28 may extend from the housing 14 distally over the elongate shaft 12. In some cases, the sterile sleeve 28 may be coupled with the hub 42 of the introducer sheath 30 and/or a hub of the repositioning sheath 32 to maintain a sterile field about the elongate shaft 12 during a medical procedure.

In some configurations of the sterile sleeve 28, the sterile sleeve 28 may include the distal head 46 and a tubular member 48 extending proximally from the head 46 to the housing 14. The tubular member 48 of the sterile sleeve 28 may be flexible and configured to have bunched locations 52 when the head 46 of the sterile sleeve 28 is adjusted proximally along the elongate shaft 12, as depicted, for example, in FIG. 2. A bunched location 52 may be a location along the sterile sleeve 28 where excess portions of the tubular member 48 may be grouped together.

The tubular member 48 of the sterile sleeve 28 may be formed from any suitable materials. Example suitable material for forming the tubular member 48 include, but are not limited to, polyurethane, thermoplastic polyurethane (TPU), polyethylene, ethylene vinyl acetate (EVA), and/or other suitable materials.

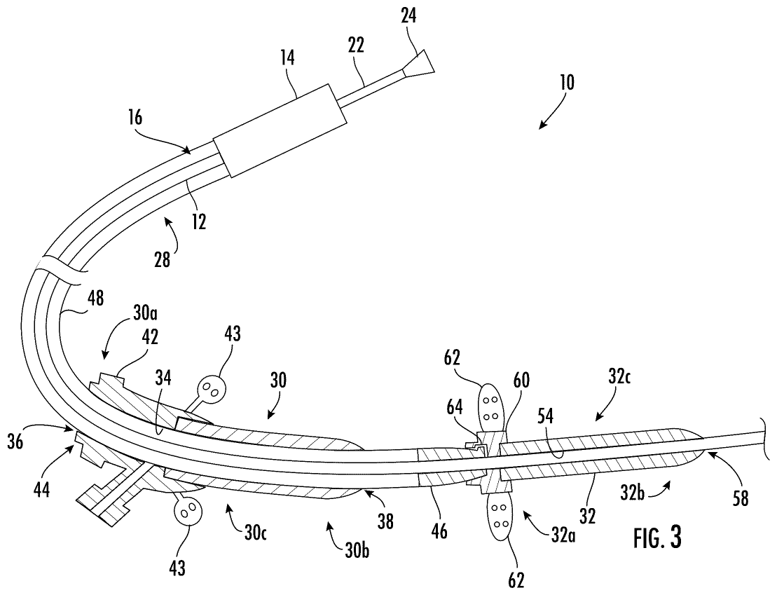

FIG. 3 depicts the catheter 10 with the proximal end of the repositioning sheath 32 distal of the distal end of the introducer sheath 30 and with the sterile sleeve 28 inserted through the introducer sheath 30. Further, a hub 60 (e.g., a hub assembly) may be at and/or coupled to the proximal end of the repositioning sheath 32 and may or may not be considered part of the repositioning sheath 32 after coupling with the repositioning sheath 32.

The hub 60 may be any suitable hub configured to couple (e.g., in a permanent or releasably/separable manner) with the repositioning sheath 32 and/or the elongate shaft 12. In some cases, the hub 60 may include one or more coupling mechanisms configured to couple to the repositioning sheath 32 and/or to elongate shaft 12 to facilitate releasably fixing the elongate shaft 12 relative to the repositioning sheath 32. Example suitable coupling mechanisms include, but are not limited to, permanent coupling mechanisms, releasable coupling mechanisms, a luer connection, a clip, a clasp, a friction fit connection, a rotational tightening mechanism, threads, and/or other suitable type of coupling mechanism. In some examples, the hub 60 may include a first coupling mechanism configured to couple with the proximal end of the repositioning sheath 32 and a second coupling mechanism (e.g., an elongate shaft lock) configured to couple with the elongate shaft 12 at a desired location along the elongate shaft 12, where the first coupling mechanism and the second coupling mechanism may be separately actuatable relative to each other, but this is not required. Further, the hub 60 may include one or more suture pads (e.g., two suture pads 62 extending from the hub 60, as depicted in FIG. 3) that may be sutured and/or otherwise coupled to a skin surface of the subject to fix the repositioning sheath 32 (and the elongate shaft 12 when coupled to the hub 60 and the repositioning sheath 32) relative to the access site of the subject.

The hub 42 of the introducer sheath 30 and/or a hub 60 of the repositioning sheath 32 may include a port for receiving and/or coupling with the head 46 of the sterile sleeve 28. In some examples, the hub 60 may include a port 64 configured to receive the head 46 of the sterile sleeve 28 within the lumen 54 to couple the sterile sleeve 28 with the repositioning sheath 32 (e.g., via a friction fit connection, a luer connection, a clip or clasp connection, threads, and/or other suitable type of connection) and create a sterile zone along the elongate shaft 12 between the proximal end of the repositioning sheath 32 and the distal end of the housing 14. Additionally or alternatively, in some examples the hub 42 may be configured to receive the head 46 of the sterile sleeve 28 within the lumen 34 and the tightening port 44 may be tightened to engage the elongate shaft 12 through the tubular member 48 of the sterile sleeve.

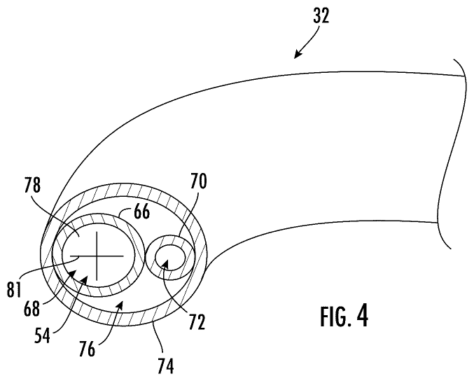

FIG. 4 depicts a schematic cross-sectioned view of an illustrative configuration of the repositioning sheath 32 independent of the other components of the catheter 10. Although not required, the repositioning sheath 32 may be a dual lumen sheath with a first tube 66 defining a first lumen 68 (e.g., the first lumen 68 may be the lumen 54, discussed above, configured to receive the elongate shaft 12 of the catheter 10), a second tube 70 extending along the first tube 66 and defining a second lumen 72 configured to receive one or more wires, and a third tube 74 extending over the first tube 66 and the second tube 70. Other suitable configurations of the repositioning sheath 32 are contemplated. In some cases, the third tube 74 may define a third lumen 76, but this is not required and the third tube 74 may be configured to (e.g., heat shrunk, etc.) fit over and fill gaps around the first tube 66 and the second tube 70.

The first tube 66, the second tube 70, and/or the third tube 74 may be formed from one or more suitable materials. Example suitable materials include, but are not limited to, polymeric and/or metallic materials. In some instances, the first tube 66, the second tube 70, and/or the third tube 74 may include one or more additional surface coatings on the interior surfaces defining the lumens 68, 72, 76 and/or the exterior surfaces, which may include, but are not limited to, silicone, polyethylene terephthalate (PET), and/or other suitable coating materials.

The first lumen 68 may include a hemostasis valve 78 (e.g., an internal hemostasis valve) extending across the first lumen 68 (e.g., the lumen 54 configured to receive the elongate shaft 12) to prevent blood from flowing through the first lumen 68 and out of the repositioning sheath 32. Additionally or alternatively, the second lumen 72 may include a hemostasis valve (not shown).

The hemostasis valve 78 may be any suitable type of valve configured to block blood flow through the first lumen 68. In some examples, the hemostasis valve 78 may be or and/or may be formed from a silicone disc with a cross-cut 81 configured to receive the elongate shaft 12 and allow the repositioning sheath 32 to pass over and seal around the elongate shaft 12. However, it is contemplated that the hemostasis valve 78 may be formed from one or more additional or alternative materials and/or include one or more other suitable cuts or openings configured to receive and seal around the elongate shaft 12.

FIG. 5 depicts a schematic cross-sectioned view of the illustrative configuration of the repositioning sheath 32 depicted in FIG. 4, with components of the catheter 10. For example, the repositioning sheath 32 may include or may be coupled with the hub 60 and the elongate shaft 12 may extend through the first lumen 68 and the hemostasis valve 78.

The hub 60 may be configured to couple with the proximal end region 32a of the repositioning sheath 32 in any suitable manner, as discussed. In some examples, the hub 60 may couple with the repositioning sheath via a clip coupling, where the hub 60 includes or forms a clip or the clip is a separate component from the hub 60. In one example, the hub 60 including a clip configuration may have a first portion 60a and a second portion 60b configured to engage one another (e.g., as depicted in FIG. 5) and couple with the repositioning sheath 32 and disengage from one another to separate from the repositioning sheath 32. In the example, the hub 60 may include one or more wings 61 that may be adjusted in a circumferential direction toward one another and adjusted radially inward or outward to engage or disengage one another to couple the hub 60 with or separate the hub 60 from the repositioning sheath 32. Other suitable coupling techniques for engaging the repositioning sheath 32 with the hub 60 are contemplated.

The sterile sleeve 28 may extend over the elongate shaft 12 to create a sterile boundary over the elongate shaft 12 from the hub 60 to the housing 14. In some cases, the head 46 of the sterile sleeve 28 may couple with the hub 60, as discussed above.

The hub 60 may be configured to receive a stylet 80 having a wire 82 and a connector 84 (e.g., a cap). The stylet 80 may be inserted into the hub 60 and the repositioning sheath 32 such that the wire 82 may extend through the second lumen 72, but this is not required and the wire 82 may extend through one or more other suitable lumens. When the wire 82 of the stylet 80 extends through the second lumen 72, the wire 82 may be configured to prevent the second lumen 72 from becoming blocked by coagulated blood, but this is not required.

The connector 84 of the stylet 80 may be configured to couple with the hub 60 with any suitable adjustable or separable coupling techniques and/or mechanisms. Example suitable coupling techniques and/or mechanisms include, but are not limited to, a luer connection, a clip, a clasp, a friction fit connection, a rotational tightening mechanism, threads, and/or other suitable types of coupling mechanisms.

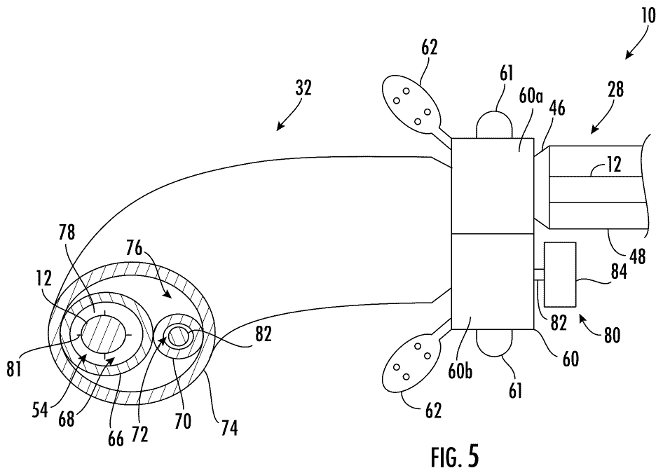

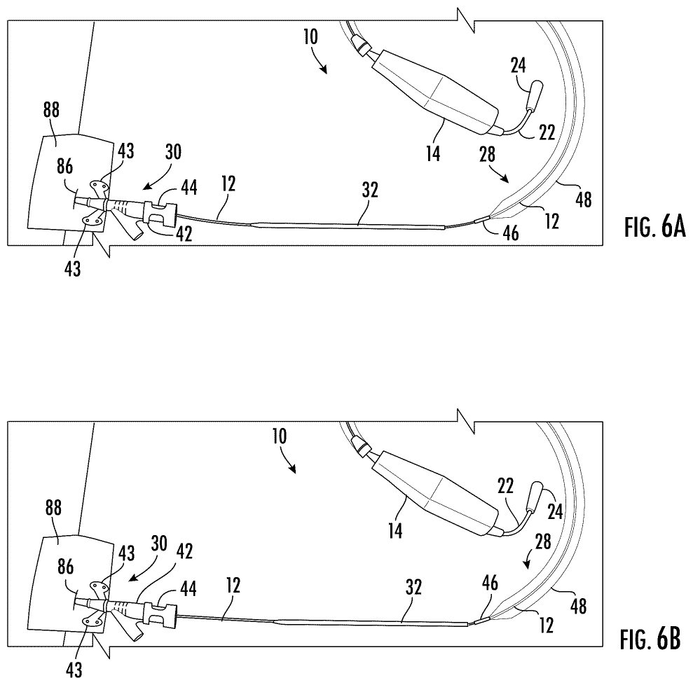

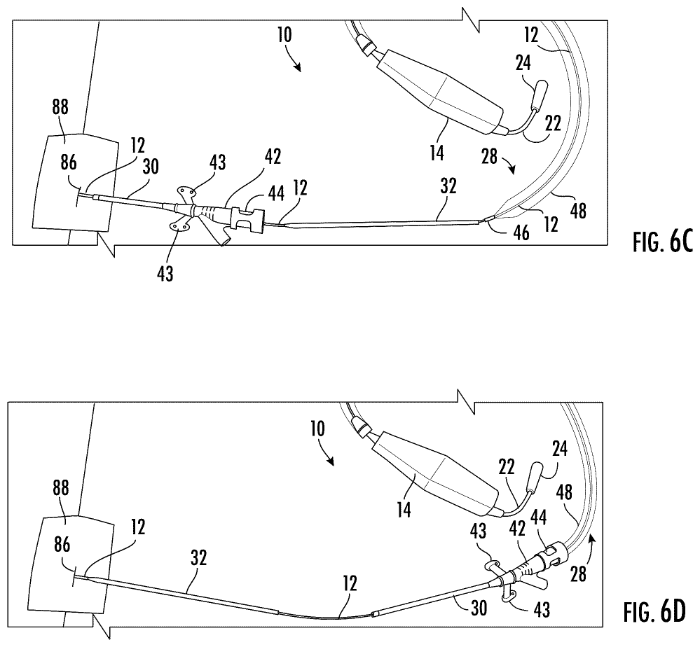

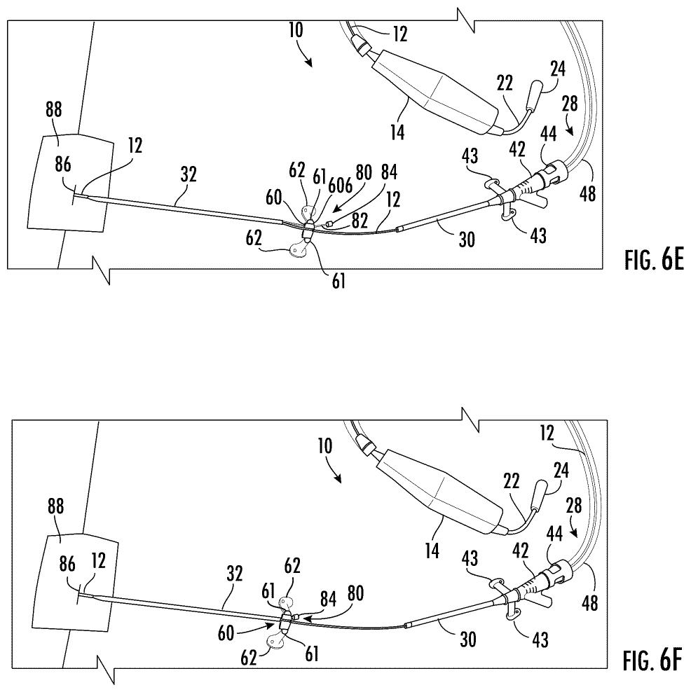

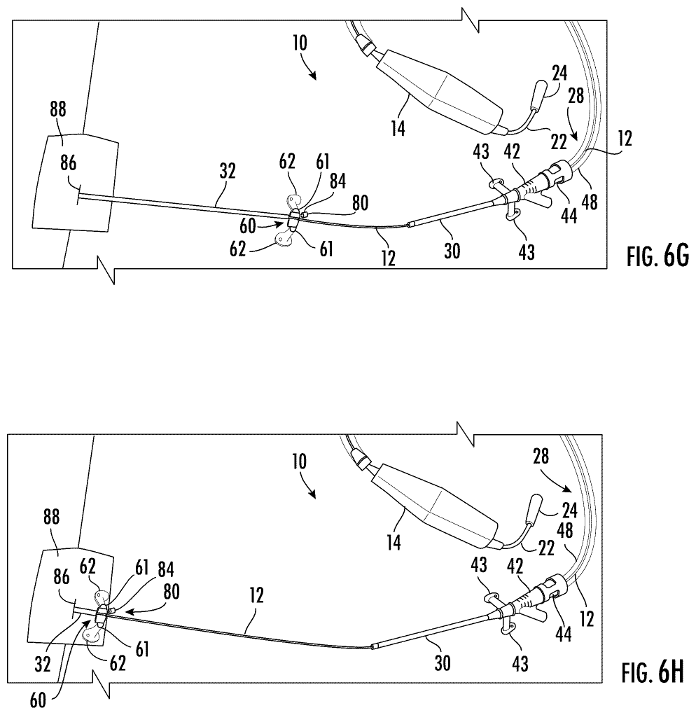

FIGS. 6A-6H schematically depict an illustrative technique for replacing the introducer sheath 30 with the repositioning sheath 32 at an access site 86 in a subject 88. Although the illustrative technique for replacing the introducer sheath 30 with the repositioning sheath 32 is discussed with respect to the components of the catheter 10, other suitable components may be utilized as desired.

FIG. 6A schematically depicts the catheter 10 inserted into the access site 86 of the subject 88, with the elongate shaft 12 extending proximal from the proximal hub 42 of the introducer sheath 30. Prior to insertion of the blood pump 50 and the elongate shaft 12 extending proximally from the blood pump 50, the introducer sheath 30 may be inserted into the access site 86 and a blood vessel (not shown) of the subject 88. Once the introducer sheath 30 has been positioned within the access site 86 and the blood vessel, the blood pump 50 and the elongate shaft 12 may be advanced distally through the introducer sheath 30 and into the blood vessel of the subject 88. During insertion of the blood pump 50 through the introducer sheath 30 and into the subject 88, the repositioning sheath 32 and the sterile sleeve 28 may remain proximal of the introducer sheath 30.

FIG. 6B schematically depicts the introducer sheath 30 positioned in the access site 86, with the repositioning sheath 32 advanced proximally (e.g., withdrawn) along the elongate shaft 12 to provide room for the introducer sheath 30 to be withdrawn proximally out of the access site 86 and along the elongate shaft 12. Further, the sterile sleeve 28 may be advanced proximally (e.g., withdrawn) along the elongate shaft 12 if needed to facilitate providing room for the introducer sheath 30 to be advanced proximally from the access site 86. However, advancing the repositioning sheath 32 proximally along the shaft 12 may be done prior to inserting the introducer sheath 30 into the access site and/or may be omitted (e.g., in instances when the repositioning sheath 32 may be inserted into the introducer sheath 30 prior to advancing the introducer sheath 30 proximally from the access site 86 and/or in other suitable instances.

FIG. 6C schematically depicts the introducer sheath 30 advanced proximally from the access site 86 (e.g., fully withdrawn from the access site 86 with the distal end of the introducer sheath 30 proximal of the access site 86). If the suture pads 43 of the hub 42 are sutured to the subject 88, the sutures may be removed from the suture pads 43 and/or the subject 88 prior to withdrawing the introducer sheath 30 from the access site 86. As depicted, after removal of the introducer sheath 30 from the access site 86, the elongate shaft 12 may extend across the access site 86 and proximal from the access site 86.

FIG. 6D schematically depicts the repositioning sheath 32 advanced along the elongate shaft 12 and fully distal of the introducer sheath 30 (e.g., with a proximal end of the repositioning sheath 32 distal of a distal end of the introducer sheath 30, such that an entirety of the repositioning sheath 32 is advanced through the introducer sheath 30). The repositioning sheath 32 may be advanced toward the access site 86 until the repositioning sheath 32 is at a suitable position proximal of the access site 86. In some cases, the hemostasis valve 78 of the repositioning sheath 32 may create friction with the elongate shaft 12 and a slight resistance may be felt as the repositioning sheath 32 is adjusted. Care may be taken to maintain a position of the elongate shaft 12 relative to access site 86 as the sterile sleeve 28, introducer sheath 30, and the repositioning sheath 32 are adjusted along the elongate shaft 12 so as to not unintentionally move a position of the blood pump 50 within the subject 88.

To facilitate positioning the repositioning sheath 32 distal of the introducer sheath 30, the introducer sheath 30 may be advanced proximally (e.g., slid and/or withdrawn in one or more other suitable manners) over the repositioning sheath 32 until a tip (e.g., a distal tip) of the repositioning sheath 32 is exposed distal of the distal end of the introducer sheath 30. Once the tip of the repositioning sheath 32 is exposed distal of the distal end of the introducer sheath 30, the distal end of the repositioning sheath 32 may be grasped and pulled toward the access site 86 over the elongate shaft 12. Other suitable techniques for advancing the introducer sheath 30 and/or the repositioning sheath 32 are contemplated.

FIG. 6E schematically depicts the repositioning sheath 32 inserted into the access site 86 and the hub 60 for coupling with the proximal end of the repositioning sheath 32 positioned over or about the elongate shaft 12. In some examples, the hub 60 may be snapped onto and/or otherwise coupled with the elongate shaft 12. Prior to, during, or after positioning the hub 60 about the elongate shaft 12, the wire 82 of the stylet 80 may be inserted into the hub 60 and into a wire lumen (e.g., the second lumen 72 discussed above and/or other suitable lumen) of the repositioning sheath 32.

FIG. 6F schematically depicts the hub 60 coupled with the proximal end of the repositioning sheath 32. Relative to a position of the hub 60 in FIG. 6F, the hub 60, which may or may not be coupled with the elongate shaft 12, may be advanced toward the repositioning sheath 32 and/or the repositioning sheath 32 may be advanced toward the hub 60 and the hub 60 may be coupled with the repositioning sheath 32 (e.g., a proximal end of the repositioning sheath 32). In some examples, the proximal end of the repositioning sheath 32 and the hub 60 may be snapped together, but this is not required and the hub 60 may be coupled with the repositioning sheath 32 in one or more other suitable manners. Once coupled, the hub 60 may be considered to be part of the repositioning sheath 32, but this is not required. Further, in some examples, an elongate shaft lock of the hub 60 may be adjusted a lock position or configuration to releasably lock the repositioning sheath 32 with or relative to the elongate shaft 12, where the elongate shaft lock may be adjusted to the lock position or configuration in any suitable manner (e.g., via a rotational lock, a snap lock, clip lock, etc.)

Prior to, during, or after coupling the hub 60 with the repositioning sheath 32, the stylet 80 may be fully advanced toward the hub 60 and the connector 84 of the stylet 80 may be coupled with the hub 60. In some examples, the connector 84 may be coupled with the hub 60 using a luer lock connection and/or a threaded connection, but this is not required and other suitable coupling techniques may be used to couple the connector 84 and the hub 60.

FIG. 6G schematically depicts the repositioning sheath 32 extending within the access site 86. In some examples, the elongate shaft lock of the hub 60 may be adjusted to an unlocked position or configuration and the repositioning sheath 32 may be advanced toward the access site 86 along the elongate shaft 12 and into the access site 86 as depicted in FIG. 6G.

FIG. 6H schematically depicts the repositioning sheath 32 inserted into the access site 86 a desired amount. In some examples, the suture pads 62 of the hub 60 may be proximate skin of the subject 88 when the repositioning sheath 32 has been inserted into the access site 86 the desired amount, but this is not required. When the suture pads 62 are proximate the skin of the subject 88, the suture pads 62 may be sutured to the subject 88. Additionally or alternatively, the elongate shaft lock may be adjusted to a lock position or configuration to fix a position of the repositioning sheath 32 relative to the elongate shaft 12.

Once the repositioning sheath 32 has been fixed relative to the elongate shaft 12 and/or the access site 86 (e.g., via suturing the suture pads to the subject 88), one or more actions may be taken to adjust the blood pump 50, apply the sterile sleeve 28 over the entire portion of the elongate shaft proximal of the repositioning sheath 32, and/or perform one or more other suitable processes. Further, additional or alternative steps are contemplated to facilitate inserting the repositioning sheath 32 into the subject 88.

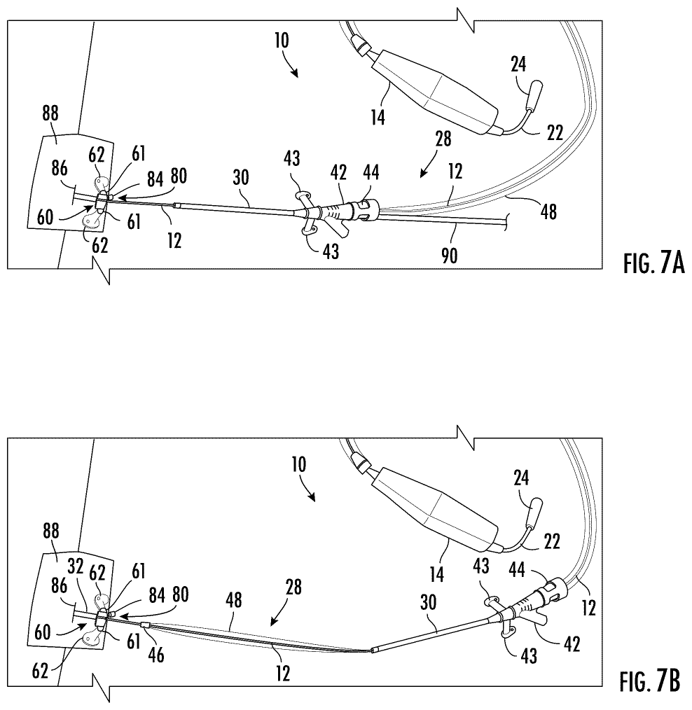

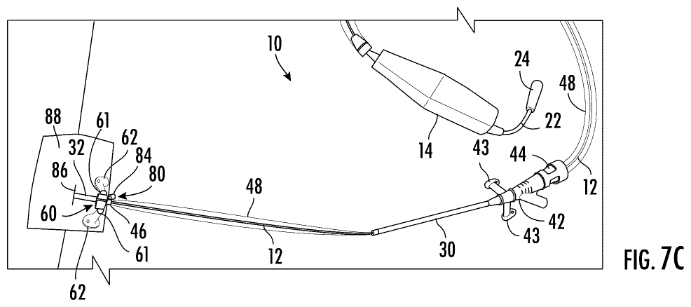

FIGS. 7A-7C schematically depict an illustrative technique for advancing the sterile sleeve 28 along the elongate shaft 12 to create a sterile corridor between the repositioning sheath 32 and the housing 14. Adjusting the sterile sleeve 28 along the elongate shaft 12 may occur prior to, during, or after positioning the repositioning sheath 32 in the access site 86. Although the illustrative technique for advancing the sterile sleeve 28 along the elongate shaft 12 is discussed with respect to the components of the catheter 10, other suitable components may be utilized as desired.

FIG. 7A schematically depicts the repositioning sheath 32 extending within the access site 86 and a distal end of the sterile sleeve 28 (e.g., the head 46 of the sterile sleeve 28) positioned within the introducer sheath 30. In some examples, a user may grasp the head 46 of the sterile sleeve 28, advance the sterile sleeve 28 along the elongate shaft 12, and position the sterile sleeve 28 at or within the introducer sheath 30 (e.g., at or within the hub 42 of the introducer sheath 30).

Although not required, a pusher rod 90 may be utilized to advance (e.g., push or otherwise pass) the sterile sleeve 28 through the introducer sheath 30 until at least a distal end (e.g., the head 46) of the sterile sleeve 28 is distal of the distal end of the introducer sheath 30. In some examples, a distal end of the pusher rod 90 may engage a proximal end of the head 46 of the sterile sleeve 28 and a force may be applied to the pusher rod 90 in the distal direction until the head 46 is positioned distally of the distal end of the introducer sheath 30.

FIG. 7B schematically depicts the distal end of the sterile sleeve 28 (e.g., the head 46 of the sterile sleeve 28) extending distally of the distal end of the introducer sheath 30. Once the sterile sleeve 28 is distal of the distal end of the introducer sheath 30, a user may grasp the head 46 and adjust the sterile sleeve 28 toward the repositioning sheath 32 (e.g., toward the hub 60 of the repositioning sheath 32) for coupling the head with the hub 60 of the repositioning sheath 32.

FIG. 7C schematically depicts the distal end of the sterile sleeve 28 (e.g., the head 46 of the sterile sleeve 28) coupled with the hub 60 of the repositioning sheath 32. A user may engage or couple the head 46 of the sterile sleeve 28 with the hub 60 of the repositioning sheath 32 in any suitable manner. In some examples, the connector 84 may be coupled with the hub 60 using a luer lock connection and/or a threaded connection, but this is not required and other suitable coupling techniques may be used to couple the head 46 and the hub 60. Once the sterile sleeve 28 is coupled with the repositioning sheath 32, a sterile barrier has been created over the elongate shaft 12 between the repositioning sheath 32 and the housing 14. In some cases, the introducer sheath 30 may be advanced fully proximally along the elongate shaft 12 to the housing 14, but this is not required.

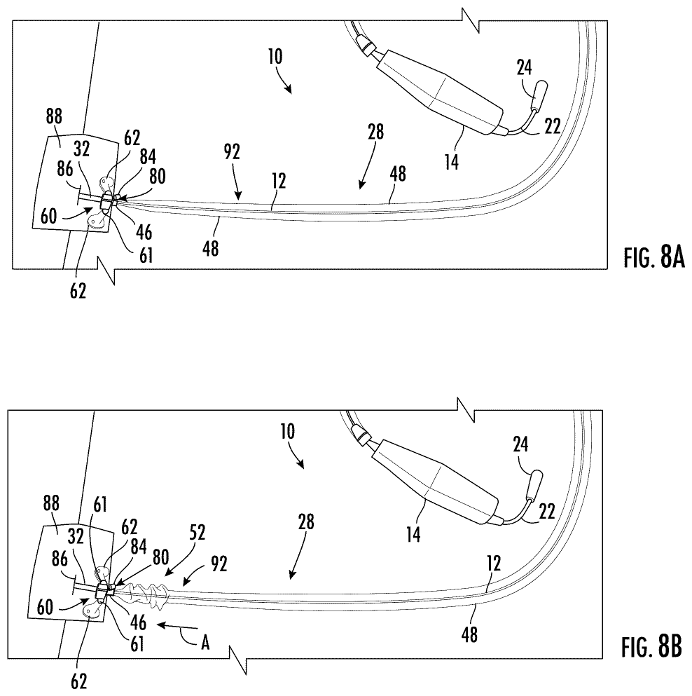

FIGS. 8A and 8B schematically depict an illustrative technique for repositioning the blood pump 50 (not shown) by adjusting the elongate shaft 12 proximal of the repositioning sheath 32. As depicted in FIGS. 8A and 8B, the introducer sheath has been advanced proximally toward the housing 14 so as to not interfere with repositioning of the blood pump 50. Although the illustrative technique for repositioning the blood pump 50 is discussed with respect to the components of the catheter 10, other suitable components may be utilized as desired.

FIG. 8A schematically depicts the sterile sleeve 28 coupled with the hub 60 at the proximal end region 32a of the repositioning sheath 32. In preparation for adjusting a position of the blood pump 50, the elongate shaft lock of the hub 60 may be opened or adjusted to an unlocked position or configuration (e.g., releasing the hub 60 from the elongate shaft 12) such that the elongate shaft 12 may adjust positions relative to the repositioning sheath 32. Further, a grasp location 92 along the sterile sleeve 28 and the elongate shaft 12 at which the elongate shaft 12 may be grasped through the sterile sleeve 28 for adjusting the blood pump 50 is depicted in FIG. 8A.

FIG. 8B schematically depicts the elongate shaft 12 and the sterile sleeve 28 advanced (e.g., adjusted) distally in the direction of arrow A. In the example adjustment of the elongate shaft 12 depicted in FIG. 8B to adjust a position of the blood pump 50, a user may grasp the elongate shaft 12 through the tubular member 48 (e.g., the flexible tubular member) of the sterile sleeve 28 at the grasp location 92 and advance both of the elongate shaft 12 and the tubular member 48 distally, which results in advancing the blood pump 50 within the subject 88 and creating a bunched location 52 distal of the grasp location 92. The blood pump 50 may be adjusted in a proximal direction by advancing the elongate shaft 12 and the tubular member 48 in the proximal direction. Once the blood pump 50 and/or the elongate shaft 12 has been adjusted to a desired location, the elongate shaft lock at the hub 60 may be adjusted to a locked position or configuration (e.g., coupling the elongate shaft 12 and the hub 60) to secure the elongate shaft 12 with or relative to the repositioning sheath 32.

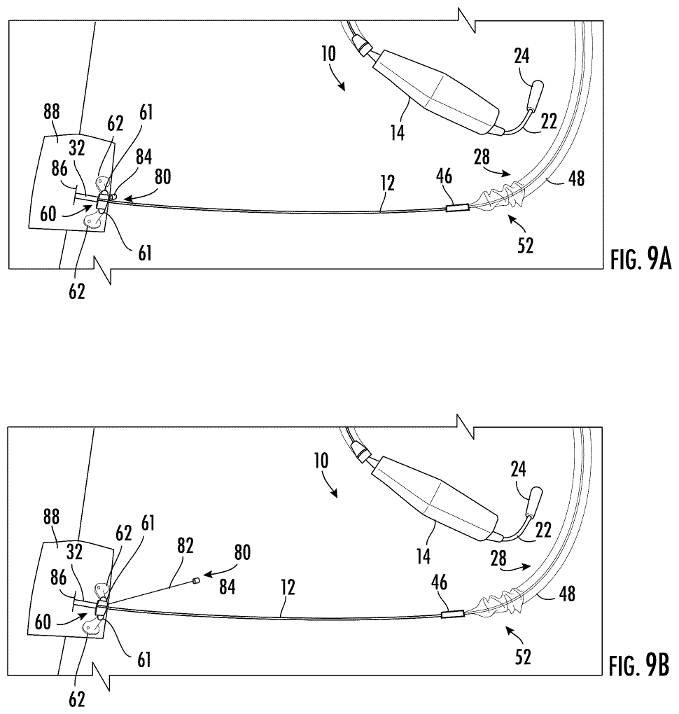

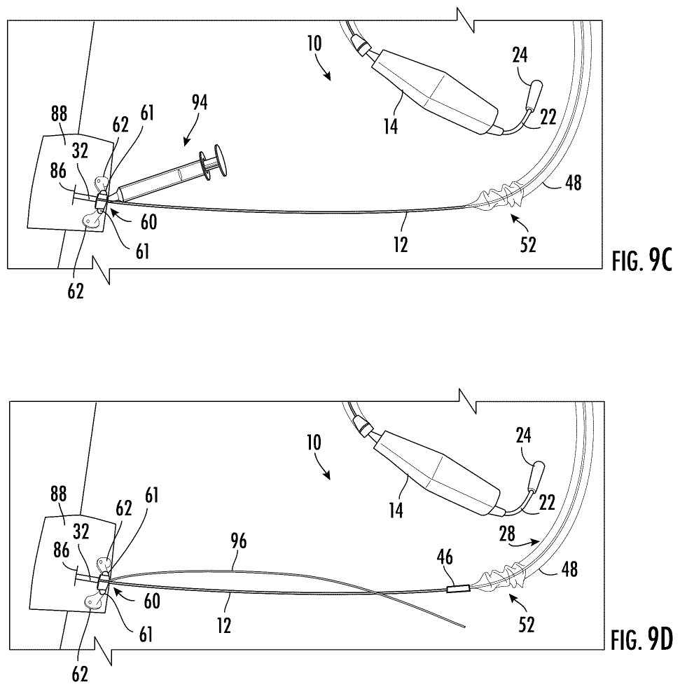

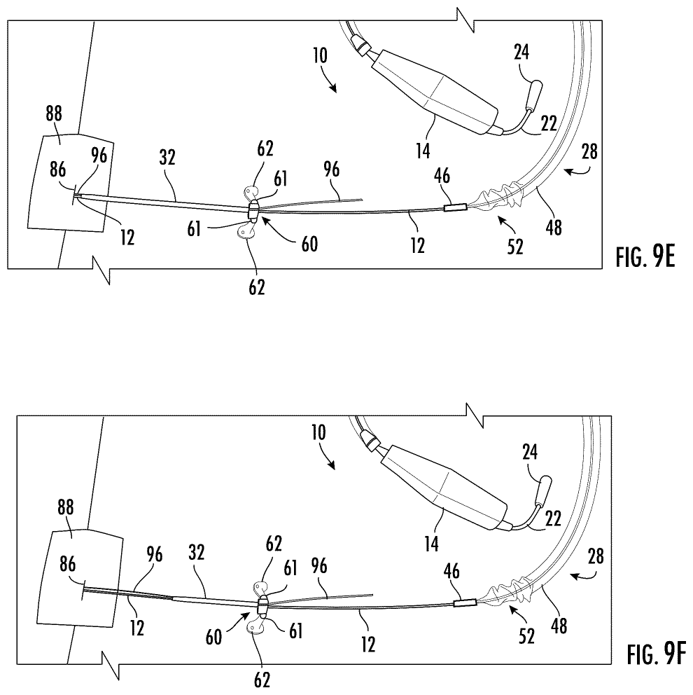

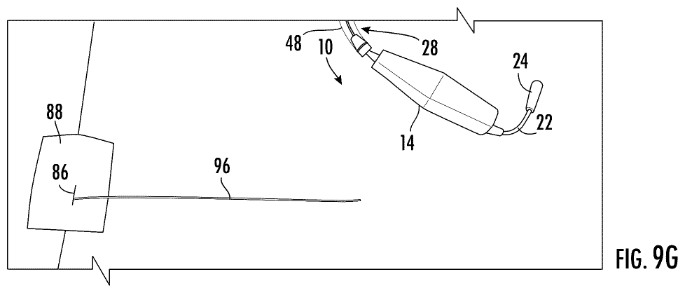

FIGS. 9A-9G schematically depict an illustrative technique for removing the blood pump 50 from the subject 88. As depicted in FIGS. 9A-9G, the introducer sheath 30 has been advanced proximally toward the housing 14 so as to not interfere with removal of the blood pump 50. Although the illustrative technique for removing the blood pump 50 is discussed with respect to the components of the catheter 10, other suitable components may be utilized as desired.

FIG. 9A schematically depicts the sterile sleeve 28 disconnected and withdrawn from the hub 60 of the repositioning sheath 32. In operation, a user may disconnect the head 46 of the sterile sleeve 28 from hub 60 and adjust the sterile sleeve 28 in the proximal direction away from the repositioning sheath 32 along the elongate shaft 12. To adjust a position of the sterile sleeve 28, the user may grasp the head 46 and/or the tubular member 48 of the sterile sleeve 28 and then move the sterile sleeve 28 in one or more directions.

FIG. 9B schematically depicts the stylet 80 being withdrawn from the hub 60 of the repositioning sheath 32. In some examples, the connector 84 may be disconnected (e.g., unscrewed, etc.) from the hub 60 and advance in a proximal direction away from the hub 60 to remove the wire 82 of the stylet 80 from the repositioning sheath 32 (e.g., from the second lumen 72 of the repositioning sheath 32).

FIG. 9C schematically depicts a syringe 94 in fluid communication with a lumen (e.g., the second lumen 72) of the repositioning sheath 32 in which the wire 82 was previously positioned. In some cases, the syringe 94 may couple with the hub 60 in the same or similar manner as the stylet 80. With the syringe 94, a user may flush (e.g., with suction) the lumen to remove any debris therein. Although the syringe 94 is depicted as being used to flush the lumen, other suitable components may be utilized to flush the lumen including, but not limited to, an electromechanical suction device and/or other suitable device.

FIG. 9D schematically depicts a wire 96 inserted into the hub 60 of the repositioning sheath 32. In some examples, the wire 96 may be inserted into the hub 60, into the lumen (e.g., the second lumen 72) of the repositioning sheath 32 in which the wire 82 of the stylet 80 was located, through the repositioning sheath 32 and the access site 86, and into the subject 88 (e.g., in a blood vessel in which the elongate shaft 12 is located). In some cases, the wire 96 may remain in the subject 88 at the access site 86 after the blood pump 50 has been removed to facilitate one more additional treatments or procedures if any are needed after the blood pump 50 has been removed from the subject 88. The wire 96 may be utilized for other suitable purposes, as desired.

The wire 96 may have any suitable diameter. In some examples, the wire 96 may have a diameter of 0.035 inches (or 0.889 millimeters), but this is not required. The wire 96 may be a guide wire, but other suitable wires are contemplated.

Once the wire 96 has been placed within the subject, elongate shaft lock of the hub 60 may be adjusted to an unlock position or configuration and the elongate shaft 12 may be advanced in a proximal direction relative to the repositioning sheath 32 to move the blood pump 50 into the descending aorta of the subject 88, while maintaining a position of the wire 96 in the subject 88. Moving the blood pump 50 into the descending aorta of the subject 88 may occur before, during, or after removing the repositioning sheath 32 from the access site 86 (e.g., as discussed with respect to FIG. 9E below). In some examples, the elongate shaft lock may remain in a locked configuration and the repositioning sheath 32 may be advanced in the proximal direction with the elongate shaft 12 to move the blood pump 50 into the descending aorta, while maintaining a position of the wire 96 in the subject 88, but this is not required.

FIG. 9E schematically depicts the repositioning sheath 32 removed from the access site 86. As the repositioning sheath 32 is removed from the access site 86, the wire 96 may be maintained within the subject 88.

FIG. 9F schematically depicts the repositioning sheath 32 further removed from the access site 86 and advanced proximally toward the housing 14. When the repositioning sheath 32 has been fully removed from the access site 86, the wire 96 may be held in place within the access site 86 (e.g., via tape, a user, etc.) and a user may fully withdraw the elongate shaft 12 and the blood pump 50 from the subject 88.

FIG. 9G schematically depicts the wire 96 remaining in the access site 86 of the subject 88 after the elongate shaft 12 and the blood pump 50 have been removed. In some examples, the access site 86 may be closed around the wire 96 to prevent leakage of body fluids (e.g., blood) through the access site 86. Closing the access site 86 may include suturing the access site closed around the wire 96 and/or one or more other suitable closing techniques.

The schematic methods and techniques discussed herein may be used together, unless expressly indicated otherwise. Further, the order of implementation of the steps discussed herein may be in other suitable orders, unless expressly indicated otherwise, and/or the methods and techniques may include one or more intervening steps discussed herein or otherwise.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A mechanical circulatory support system, the system comprising:
a blood pump configured to pump blood from a ventricle of a heart of a patient to vasculature of the patient;
a housing;
an elongate shaft coupled with the blood pump and extending proximally from the blood pump to the housing;
a first sheath configured to extend over the elongate shaft distal of the housing, the first sheath has a lumen with an inner diameter; and
a second sheath configured to extend over the elongate shaft distal of the housing, the second sheath has an outer diameter that is less than the inner diameter of the lumen, and
wherein the first sheath and the second sheath are configured to longitudinally adjust over the elongate shaft and an entirety of the second sheath is configured to adjust through the first sheath.

2. The system of claim 1, wherein the first sheath is an introducer sheath configured to allow the blood pump to pass through the lumen.

3. The system of claim 1, wherein the second sheath is a dual lumen sheath.

4. The system of claim 3, wherein the dual lumen sheath comprises a first tube defining a first lumen, a second tube extending along the first tube and defining a second lumen, and a third tube extending over the first tube and the second tube.

5. The system of claim 3, the system further comprises:
a wire, and
wherein the dual lumen sheath defines a first lumen configured to receive the elongate shaft and a second lumen configured to receive the wire.

6. The system of claim 1, wherein the second sheath includes an internal hemostasis valve.

7. The system of claim 1, further comprising:
a hub assembly configured to couple with a proximal end of the second sheath.

8. The system of claim 7, further comprising:
a wire with a connector at a proximal end of the wire, and
wherein the connector is configured to couple with the hub assembly.

9. The system of claim 7, wherein the hub assembly is configured to releasably engage the elongate shaft to fix the second sheath with respect to the elongate shaft.

10. The system of claim 7, further comprising:
a sleeve extending over the elongate shaft distal of the housing, the sleeve has a distal end configured to couple with the hub assembly.

11. A method of adjusting a tube assembly for a mechanical circulatory support system, the method comprising:
advancing a blood pump and an elongate shaft extending proximally from the blood pump through a first sheath;
advancing the first sheath along the elongate shaft in a proximal direction;
advancing an entirety of a second sheath through the first sheath; and
coupling a hub assembly with a proximal end of the second sheath.

12. The method of claim 11, further comprising:
coupling the hub assembly with the elongate shaft; and
advancing the hub assembly coupled with the elongate shaft toward the proximal end of the second sheath to couple the hub assembly with the proximal end of the second sheath.

13. The method of claim 11, wherein the second sheath has a first lumen configured to receive the elongate shaft and a second lumen, the method further comprising:
inserting a wire through the second lumen.

14. The method of claim 13, further comprising:
coupling a connector at a proximal end of the wire to the hub assembly.

15. The method of claim 11, further comprising:
coupling the hub assembly with the elongate shaft;
releasing the hub assembly from the elongate shaft;
adjusting the elongate shaft relative to the hub assembly and the second sheath; and
re-coupling the hub assembly with the elongate shaft.

16. The method of claim 11, further comprising:
passing a distal end of a sleeve through the first sheath; and
coupling the distal end of the sleeve with the hub assembly.

17. The method of claim 16, further comprising:
adjusting the elongate shaft relative to the hub assembly and the second sheath, and
wherein adjusting the elongate shaft comprises engaging the elongate shaft through the sleeve and advancing the elongate shaft proximally or distally.

18. A mechanical circulatory support system, the system comprising:
a blood pump configured to pump blood from a ventricle of a heart of a patient to vasculature of the patient;
an elongate shaft coupled with the blood pump and extending proximally from the blood pump;
a repositioning sheath extending over the elongate shaft; and
a hub assembly configured to couple to a proximal end of the repositioning sheath, and
wherein the hub assembly is configured to lock the repositioning sheath at a position relative to the elongate shaft.

19. The system of claim 18, further comprising:
a sterile sleeve extending over the elongate shaft, and
wherein the hub assembly is configured to couple with a distal end of the sterile sleeve.

20. The system of claim 18, further comprising:
a stylet having a connector and a wire extending distally from the connector, and
wherein:
the repositioning sheath has a first lumen configured to receive the elongate shaft and a second lumen configured to receive the wire, and
the hub assembly is configured to couple with the connector.
